SEQUENCE LISTING (65 sequences)

SEQ ID NO: 1 - DNA, 9 bp, Artificial Sequence
  Description of Artificial Sequence Synthetic oligonucleotide
caaaaaaag

SEQ ID NO: 2 - DNA, 9 bp, Artificial Sequence
  Description of Artificial Sequence Synthetic oligonucleotide
caaaagaag

SEQ ID NO: 3 - DNA, 9 bp, Artificial Sequence
  Description of Artificial Sequence Synthetic oligonucleotide
cagaagaag

SEQ ID NO: 4 - DNA, 9 bp, Artificial Sequence
  Description of Artificial Sequence Synthetic oligonucleotide
caaaagaaa

SEQ ID NO: 5 - DNA, 9 bp, Artificial Sequence
  Description of Artificial Sequence Synthetic oligonucleotide
cagaaaaaa

SEQ ID NO: 6 - protein, 3 aa, Artificial Sequence
  Description of Artificial Sequence Synthetic peptide
QKK

SEQ ID NO: 7 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 8 - protein, 5 aa, Homo sapiens
SLKGS

SEQ ID NO: 9 - protein, 309 aa, Artificial Sequence
  Description of Artificial Sequence Synthetic polypeptide
CDLPQTHSLGSHHHHHHGSVVAPPAPIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 10 - protein, 283 aa, Artificial Sequence
  Description of Artificial Sequence Synthetic polypeptide
IVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 11 - protein, 284 aa, Artificial Sequence
  Description of Artificial Sequence Synthetic polypeptide
PIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 12 - protein, 285 aa, Artificial Sequence
  Description of Artificial Sequence Synthetic polypeptide
XPIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 13 - protein, 285 aa, Artificial Sequence
  Description of Artificial Sequence Synthetic polypeptide
APIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 14 - protein, 306 aa, Artificial Sequence
  Description of Artificial Sequence Synthetic polypeptide
HHHHHHXXXXXXXXXXXXXXXXXIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 15 - protein, 4 aa, Artificial Sequence
  Description of Artificial Sequence Synthetic linker peptide
GSAP

SEQ ID NO: 16 - protein, 4 aa, Artificial Sequence
  Description of Artificial Sequence Synthetic linker peptide
GSGP

SEQ ID NO: 17 - protein, 4 aa, Artificial Sequence
  Description of Artificial Sequence Synthetic linker peptide
GSSP

SEQ ID NO: 18 - protein, 4 aa, Artificial Sequence
  Description of Artificial Sequence Synthetic linker peptide
GSPP

SEQ ID NO: 19 - protein, 4 aa, Artificial Sequence
  Description of Artificial Sequence Synthetic linker peptide
GGGS

SEQ ID NO: 20 - protein, 5 aa, Artificial Sequence
  Description of Artificial Sequence Synthetic linker peptide
GGGGS

SEQ ID NO: 21 - protein, 8 aa, Artificial Sequence
  Description of Artificial Sequence Synthetic linker peptide
GGGSGGGS

SEQ ID NO: 22 - protein, 10 aa, Artificial Sequence
  Description of Artificial Sequence Synthetic linker peptide
GGGGSGGGGS

SEQ ID NO: 23 - protein, 12 aa, Artificial Sequence
  Description of Artificial Sequence Synthetic linker peptide
GGGSGGGSGGGS

SEQ ID NO: 24 - protein, 15 aa, Artificial Sequence
  Description of Artificial Sequence Synthetic linker peptide
GGGGSGGGGSGGGGS

SEQ ID NO: 25 - protein, 6 aa, Artificial Sequence
  Description of Artificial Sequence Synthetic linker peptide
GGGSAP

SEQ ID NO: 26 - protein, 6 aa, Artificial Sequence
  Description of Artificial Sequence Synthetic linker peptide
GGGSGP

SEQ ID NO: 27 - protein, 6 aa, Artificial Sequence
  Description of Artificial Sequence Synthetic linker peptide
GGGSSP

SEQ ID NO: 28 - protein, 6 aa, Artificial Sequence
  Description of Artificial Sequence Synthetic linker peptide
GGGSPP

SEQ ID NO: 29 - protein, 7 aa, Artificial Sequence
  Description of Artificial Sequence Synthetic linker peptide
GGGGSAP

SEQ ID NO: 30 - protein, 7 aa, Artificial Sequence
  Description of Artificial Sequence Synthetic linker peptide
GGGGSGP

SEQ ID NO: 31 - protein, 7 aa, Artificial Sequence
  Description of Artificial Sequence Synthetic linker peptide
GGGGSSP

SEQ ID NO: 32 - protein, 7 aa, Artificial Sequence
  Description of Artificial Sequence Synthetic linker peptide
GGGGSPP

SEQ ID NO: 33 - protein, 10 aa, Artificial Sequence
  Description of Artificial Sequence Synthetic linker peptide
GGGSGGGSAP

SEQ ID NO: 34 - protein, 10 aa, Artificial Sequence
  Description of Artificial Sequence Synthetic linker peptide
GGGSGGGSGP

SEQ ID NO: 35 - protein, 10 aa, Artificial Sequence
  Description of Artificial Sequence Synthetic linker peptide
GGGSGGGSSP

SEQ ID NO: 36 - protein, 10 aa, Artificial Sequence
  Description of Artificial Sequence Synthetic linker peptide
GGGSGGGSPP

SEQ ID NO: 37 - protein, 14 aa, Artificial Sequence
  Description of Artificial Sequence Synthetic linker peptide
GGGSGGGSGGGSAP

SEQ ID NO: 38 - protein, 14 aa, Artificial Sequence
  Description of Artificial Sequence Synthetic linker peptide
GGGSGGGSGGGSGP

SEQ ID NO: 39 - protein, 14 aa, Artificial Sequence
  Description of Artificial Sequence Synthetic linker peptide
GGGSGGGSGGGSSP

SEQ ID NO: 40 - protein, 14 aa, Artificial Sequence
  Description of Artificial Sequence Synthetic linker peptide
GGGSGGGSGGGSPP

SEQ ID NO: 41 - protein, 7 aa, Artificial Sequence
  Description of Artificial Sequence Synthetic linker peptide
GGGGSAP

SEQ ID NO: 42 - protein, 7 aa, Artificial Sequence
  Description of Artificial Sequence Synthetic linker peptide
GGGGSGP

SEQ ID NO: 43 - protein, 7 aa, Artificial Sequence
  Description of Artificial Sequence Synthetic linker peptide
GGGGSSP

SEQ ID NO: 44 - protein, 7 aa, Artificial Sequence
  Description of Artificial Sequence Synthetic linker peptide
GGGGSPP

SEQ ID NO: 45 - protein, 12 aa, Artificial Sequence
  Description of Artificial Sequence Synthetic linker peptide
GGGGSGGGGSAP

SEQ ID NO: 46 - protein, 12 aa, Artificial Sequence
  Description of Artificial Sequence Synthetic linker peptide
GGGGSGGGGSGP

SEQ ID NO: 47 - protein, 12 aa, Artificial Sequence
  Description of Artificial Sequence Synthetic linker peptide
GGGGSGGGGSSP

SEQ ID NO: 48 - protein, 12 aa, Artificial Sequence
  Description of Artificial Sequence Synthetic linker peptide
GGGGSGGGGSPP

SEQ ID NO: 49 - protein, 17 aa, Artificial Sequence
  Description of Artificial Sequence Synthetic linker peptide
GGGGSGGGGSGGGGSAP

SEQ ID NO: 50 - protein, 17 aa, Artificial Sequence
  Description of Artificial Sequence Synthetic linker peptide
GGGGSGGGGSGGGGSGP

SEQ ID NO: 51 - protein, 17 aa, Artificial Sequence
  Description of Artificial Sequence Synthetic linker peptide
GGGGSGGGGSGGGGSSP

SEQ ID NO: 52 - protein, 17 aa, Artificial Sequence
  Description of Artificial Sequence Synthetic linker peptide
GGGGSGGGGSGGGGSPP

SEQ ID NO: 53 - protein, 12 aa, Artificial Sequence
  Description of Artificial Sequence Synthetic peptide
VARRNGTVQTES

SEQ ID NO: 54 - protein, 9 aa, Artificial Sequence
  Description of Artificial Sequence Synthetic peptide
CDLPQTHSL

SEQ ID NO: 55 - protein, 6 aa, Artificial Sequence
  Description of Artificial Sequence Synthetic 6xHis tag
HHHHHH

SEQ ID NO: 56 - protein, 7 aa, Artificial Sequence
  Description of Artificial Sequence Synthetic peptide
VVAPPAP

SEQ ID NO: 57 - protein, 4 aa, Artificial Sequence
  Description of Artificial Sequence Synthetic peptide
PIVN

SEQ ID NO: 58 - protein, 5 aa, Artificial Sequence
  Description of Artificial Sequence Synthetic peptide
XPIVN

SEQ ID NO: 59 - protein, 5 aa, Artificial Sequence
  Description of Artificial Sequence Synthetic peptide
APIVN

SEQ ID NO: 60 - DNA, 100 bp, Artificial Sequence
  Description of Artificial Sequence Synthetic polynucleotide
aagttaaagcgaaagtgcagccgtacctggatgacttccaaaaaaagtggcaagaagaaatggaactgtacagacagaaagttgagccgctgcgtgcaga

SEQ ID NO: 61 - protein, 33 aa, Artificial Sequence
  Description of Artificial Sequence Synthetic polypeptide
EVKAKVQPYLDDFQKKWQEEMELYRQKVEPLRA

SEQ ID NO: 62 - protein, 33 aa, Artificial Sequence
  Description of Artificial Sequence Synthetic polypeptide
KLKRKCSRTWMTSKKSGKKKWNCTDRKLSRCVQ

SEQ ID NO: 63 - protein, 8 aa, Artificial Sequence
  Description of Artificial Sequence Synthetic peptide
SESAAVPG

SEQ ID NO: 64 - protein, 16 aa, Artificial Sequence
  Description of Artificial Sequence Synthetic peptide
LPKKVARRNGTVQTES

SEQ ID NO: 65 - protein, 5 aa, Artificial Sequence
  Description of Artificial Sequence Synthetic peptide
AAACR

(19) United States
(12) Patent Application Publication
(10) Pub. No.: US 2015/0064746 A1
(43) Pub. Date: Mar. 5, 2015

(54) METHOD FOR REDUCTION OF 1->3 READING FRAME SHIFTS

(71) Applicant: Hoffmann-La Roche Inc., Little Falls, NJ (US)

(72) Inventors: Adelbert Grossmann, Eglfing (DE); Friederike Hesse, Muenchen (DE); Erhard Kopetzki, Penzberg (DE); Wilma Lau, Muenchen (DE); Christian Schantz, Penzberg (DE)

(73) Assignee: HOFFMANN-LA ROCHE INC., Little Falls, NJ (US)

(21) Appl. No.: 14/473,367

(22) Filed: Aug. 29, 2014

Related U.S. Application Data
(63) Continuation of application No. PCT/EP2013/053753, filed on Feb. 26, 2013.

(30) Foreign Application Priority Data
Feb. 29, 2012 (EP) 12157513.8
Apr. 2, 2012 (EP) 12162814.3

(51) Int. Cl. C07K 14/775; C07K 14/47

(52) U.S. Cl. CPC C07K 14/775; C07K 14/4726; C07K 2319/00
USPC 435/69.6

(57) ABSTRACT

Herein is reported a method for the recombinant production of a polypeptide, which comprises the tripeptide QKK, characterized in that the method comprises the step of recovering the polypeptide from the cells or the cultivation medium of a cultivation of a cell comprising a nucleic acid encoding the polypeptide and thereby producing the polypeptide, whereby the tripeptide QKK comprised in the polypeptide is encoded by the oligonucleotide cag aaa aaa or the oligonucleotide caa aag aaa.

### CROSS REFERENCE TO RELATED APPLICATIONS

This application is a continuation of International Application No. PCT/EP2013/053753 having an international filing date of Feb. 26, 2013, the entire contents of which are incorporated herein by reference, and which claims benefit under 35 U.S.C. §119 to European Patent Application Nos. 12157513.8 filed Feb. 29, 2012 and 12162814.3 filed Apr. 2, 2012.

### SEQUENCE LISTING

The instant application contains a Sequence Listing submitted via EFS-Web and hereby incorporated by reference in its entirety. Said ASCII copy, created on Aug. 25, 2014, is named P4892C1SeqList.txt, and is 28,713 bytes in size.

### FIELD OF THE INVENTION

The current invention is in the field of recombinant polypeptide production. It is reported herein a method for recombinantly producing a polypeptide with reduced by-product content wherein the reduction of the by-product content is achieved by a modification of the encoding nucleic acid that reduces frameshifts during the translation or transcription process.

### BACKGROUND OF THE INVENTION

Proteins play an important role in today's medical portfolio. For human application every pharmaceutical substance has to meet distinct criteria. To ensure the safety of biopharmaceutical agents to humans nucleic acids, viruses, and host cell proteins, which would cause severe harm, have to be removed especially. To meet the regulatory specification one or more purification steps have to follow the manufacturing process.

Recombinant polypeptides can be produced e.g. by prokaryotic cells such as E. coli. The recombinantly produced polypeptide accounts for the majority of the prokaryotic cell's polypeptide content and is often deposited as insoluble aggregate, i.e. as a so called inclusion body, within the prokaryotic cell. For the isolation of the recombinant polypeptide the cells have to be disintegrated and the recombinant polypeptide contained in the inclusion bodies has to be solubilized after the separation of the inclusion bodies from the cell debris. For the solubilization chaotropic reagents, such as urea or guanidinium chloride, are used. To cleave disulfide bonds reducing agents, especially under alkaline conditions, such as dithioerythritol, dithiothreitol, or β-mercaptoethanol are added. After the solubilization of the aggregated polypeptide the globular structure of the recombinant polypeptide, which is essential for the biological activity, has to be reestablished. During this so called renaturation process the concentration of the denaturing agents is (slowly) reduced, e.g. by dialysis against a suited buffer, which allows the denatured polypeptide to refold into its biologically active structure. After renaturation the recombinant polypeptide is purified to a purity acceptable for the intended use. For example, for the use as a therapeutic protein a purity of more than 90% has to be established.

Recombinantly produced polypeptides are normally accompanied by nucleic acids, endotoxins, and/or polypeptides from the producing cell. Beside the host cell derived by-products also polypeptide-derived by-products are present in a crude polypeptide preparation. Among others shortened variants of the polypeptide of interest can be present.

In WO 95/25786 the production of human apolipoprotein AI in a bacterial expression system is reported. Karathanasis, S. K., et al., report the isolation and characterization of the human apolipoprotein A-1 gene (Proc. Natl. Acad. Sci. USA 80 (1983) 6147-6151). Sequences that direct significant levels of frameshifting are frequent in coding regions of Escherichia coli are reported by Gurvich, O. L., et al. in the EMBO Journal (22 (2003) 5941-5950). Graversen, J. H., et al., report that the trimerization of apolipoprotein A-1 retards plasma clearance and preserves anti-atherosclerotic properties (J. Cardiovascular Pharmacology 51 (2008) 170-177).

### SUMMARY OF THE INVENTION

It has been found that the oligonucleotide that encodes the tripeptide QKK can be the point of a 1->3 frameshift during the transcription or translation process of a nucleic acid that encodes a polypeptide which comprises the tripeptide QKK. Due to the occurrence of the frameshift a nonsense polypeptide with a not-encoded amino acid sequence is produced.

Thus, herein is reported as one aspect a method for the recombinant production of a polypeptide, which comprises the tripeptide QKK (SEQ ID NO: 06), characterized in that the method comprises the following step:
recovering the polypeptide from the cells or the cultivation medium of a cultivation of a cell comprising a nucleic acid encoding the polypeptide and thereby producing the polypeptide, whereby the tripeptide QKK comprised in the polypeptide is encoded by the oligonucleotide cag aag aag (SEQ ID NO: 03), or the oligonucleotide caa aag aaa (SEQ ID NO: 04), or the oligonucleotide cag aaa aaa (SEQ ID NO: 05).

In one embodiment the tripeptide QKK comprised in the polypeptide is encoded by the oligonucleotide caa aag aaa (SEQ ID NO: 04) or the oligonucleotide cag aaa aaa (SEQ ID NO: 05).

One aspect as reported herein is a nucleic acid encoding a polypeptide that comprises the tripeptide QKK in its amino acid sequence whereby the tripeptide QKK is encoded by the oligonucleotide cag aag aag (SEQ ID NO: 03), or the oligonucleotide caa aag aaa (SEQ ID NO: 04), or the oligonucleotide cag aaa aaa (SEQ ID NO: 05).

One aspect as reported herein is a nucleic acid encoding a polypeptide that comprises the tripeptide QKK in its amino acid sequence whereby the tripeptide QKK is encoded by the oligonucleotide caa aag aaa (SEQ ID NO: 04) or the oligonucleotide cag aaa aaa (SEQ ID NO: 05).

One aspect as reported herein is a cell comprising a nucleic acid as reported herein.

One aspect as reported herein is the use of the oligonucleotide cag aag aag (SEQ ID NO: 03), or the oligonucleotide caa aag aaa (SEQ ID NO: 04), or the oligonucleotide cag aaa aaa (SEQ ID NO: 05) for encoding the tripeptide QKK comprised in a polypeptide to be expressed in E. coli.

One aspect as reported herein is the use of the oligonucleotide caa aag aaa (SEQ ID NO: 04) or the oligonucleotide cag aaa aaa (SEQ ID NO: 05) for encoding the tripeptide QKK comprised in a polypeptide to be expressed in E. coli.

In the following embodiments of all aspects as reported herein are specified.

In one embodiment the tripeptide QKK is encoded by the oligonucleotide caa aag aaa (SEQ ID NO: 04).

In one embodiment the tripeptide QKK is encoded by the oligonucleotide cag aaa aaa (SEQ ID NO: 05).

In one embodiment the (full length) polypeptide comprises about 50 amino acid residues to about 500 amino acid residues. In one embodiment the (full length) polypeptide comprises about 100 amino acid residues to about 400 amino acid residues. In one embodiment the (full length) polypeptide comprises about 250 amino acid residues to about 350 amino acid residues.

In one embodiment the cell is a prokaryotic cell. In one embodiment the prokaryotic cell is an E. coli cell, or a bacillus cell.

In one embodiment the cell is a eukaryotic cell. In one embodiment the cell is a CHO cell, or a HEK cell, or a BHK cell, or a NS0 cell, or a SP2/0 cell, or a yeast cell.

In one embodiment the polypeptide is a hetero-multimeric polypeptide. In one embodiment the polypeptide is an antibody or an antibody fragment.

In one embodiment the polypeptide is a homo-multimeric polypeptide. In one embodiment the polypeptide is a homo-dimer or a homo-trimer.

In one embodiment the polypeptide is human apolipoprotein A-I or a variant thereof or a fusion polypeptide comprising it, whereby the variant or the fusion polypeptide shows in vitro and in vivo the function of human apolipoprotein A-I. In one embodiment the apolipoprotein A-I variant has the amino acid sequence selected from the group of SEQ ID NO: 09 to SEQ ID NO: 14.

### DETAILED DESCRIPTION OF THE INVENTION

### Definitions

The term “amino acid” denotes the group of carboxy α-amino acids, which directly or in form of a precursor can be encoded by nucleic acid. The individual amino acids are encoded by nucleic acids consisting of three nucleotides, so called codons or base-triplets. Each amino acid is encoded by at least one codon. The encoding of the same amino acid by different codons is known as “degeneration of the genetic code”. The term “amino acid” denotes the naturally occurring carboxy α-amino acids and comprises alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The term “apolipoprotein A-I” denotes an amphiphilic, helical polypeptide with protein-lipid and protein-protein interaction properties. Apolipoprotein A-I is synthesized by the liver and small intestine as prepro-apolipoprotein of 267 amino acid residues which is secreted as a pro-apolipoprotein that is cleaved to the mature polypeptide having 243 amino acid residues. Apolipoprotein A-I consists of 6 to 8 different amino acid repeats consisting each of 22 amino acid residues separated by a linker moiety which is often proline, and in some cases consists of a stretch made up of several residues. An exemplary human apolipoprotein A-I amino acid sequence is reported in GenPept database entry NM-000039 or database entry X00566; GenBank NP-000030.1 (gi 4557321). Of human apolipoprotein A-I (SEQ ID NO: 07) naturally occurring variants exist, such as P27H, P27R, P28R, R34L, G50R, L84R, D113E, A-A119D, D127N, deletion of K131, K131M, W132R, E133K, R151C (amino acid residue 151 is changed from Arg to Cys, apolipoprotein A-I-Paris), E160K, E163G, P167R, L168R, E171V, P189R, R197C (amino acid residue 173 is change from Arg to Cys, apolipoprotein A-I-Milano) and E222K. Also included are variants that have conservative amino acid modifications.

The term “codon” denotes an oligonucleotide consisting of three nucleotides that encodes a defined amino acid. Due to the degeneracy of the genetic code some amino acids are encoded by more than one codon. These different codons encoding the same amino acid have different relative usage frequencies in individual host cells. Thus, a specific amino acid can be encoded by a group of different codons. Likewise the amino acid sequence of a polypeptide can be encoded by different nucleic acids. Therefore, a specific amino acid can be encoded by a group of different codons, whereby each of these codons has a usage frequency within a given host cell.

TABLE
Escherichia Coli codon usage (codon | encoded
amino acid | usage frequency [%])

| | | | | | | | | | | | |
|---|---|---|---|---|---|---|---|---|---|---|---|
| TTT | F | 58 | TCT | S | 17 | TAT | Y | 59 | TGT | C | 46 |
| TIC | F | 42 | TCC | S | 15 | TAC | Y | 41 | TGC | C | 54 |
| TTA | L | 14 | TCA | S | 14 | TAA | * | 61 | TGA | * | 30 |
| TTG | L | 13 | TCG | S | 14 | TAG | * | 9 | TGG | W | 100 |
| CTT | L | 12 | CCT | P | 18 | CAT | H | 57 | CGT | R | 36 |
| CTC | L | 10 | CCC | P | 13 | CAC | H | 43 | CGC | R | 36 |
| CTA | L | 4 | CCA | P | 20 | CAA | Q | 34 | CGA | R | 7 |
| CTG | L | 47 | CCG | P | 49 | CAG | Q | 66 | CGG | R | 11 |
| ATT | I | 49 | ACT | T | 19 | AAT | N | 49 | AGT | S | 16 |
| ATC | I | 39 | ACC | T | 40 | AAC | N | 51 | AGC | S | 25 |
| ATA | I | 11 | ACA | T | 17 | AAA | K | 74 | AGA | R | 7 |
| ATG | M | 100 | ACG | T | 25 | AAG | K | 26 | AGG | R | 4 |
| GTT | V | 28 | GCT | A | 18 | GAT | D | 63 | GGT | G | 35 |
| GTC | V | 20 | GCC | A | 26 | GAC | D | 37 | GGC | G | 37 |
| GTA | V | 17 | GCA | A | 23 | GAA | E | 68 | GGA | G | 13 |
| GTG | V | 35 | GCG | A | 33 | GAG | E | 32 | GGG | G | 15 |

Exemplary changes are provided in the following Table under the heading of “exemplary substitutions”. Conservative substitutions are shown in the following Table under the heading of “preferred substitutions” and as further described below in reference to amino acid side chain classes.

TABLE

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Non-conservative substitutions will entail exchanging a member of one of these classes for another class.

The term “conservative amino acid modification” denotes modifications of the amino acid sequence which do not affect or alter the characteristics of the polypeptide. Modifications can be introduced by standard techniques known in the art, such as site-directed mutagenesis and PCR-mediated mutagenesis. Conservative amino acid modifications include ones in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine, tryptophan), non-polar side chains (e.g. alanine, valine, leucine, isoleucine, proline, phenylalanine, methionine), beta-branched side chains (e.g. threonine, valine, isoleucine), and aromatic side chains (e.g. tyrosine, phenylalanine, tryptophan, histidine).

The term “variant of a polypeptide” denotes a polypeptide which differs in amino acid sequence from a “parent” polypeptide's amino acid sequence by up to ten, in one embodiment from about two to about five, additions, deletions, and/or substitutions. Amino acid sequence modifications can be performed by mutagenesis based on molecular modeling as described by Riechmann, L., et al., Nature 332 (1988) 323-327, and Queen, C., et al., Proc. Natl. Acad. Sci. USA 86 (1989) 10029-10033.

The homology and identity of different amino acid sequences may be calculated using well known algorithms such as BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, or BLOSUM 90. In one embodiment the algorithm is BLOSUM 30.

The terms “host cell”, “host cell line”, and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages.

Progeny may not be completely identical in nucleic acid content to a parent cell, but may contain mutations. Mutant progeny that have the same function or biological activity as screened or selected for in the originally transformed cell are included herein.

The terms “nucleic acid” and “nucleic acid sequence” denote a polymeric molecule consisting of the individual nucleotides (also called bases) ‘a’, ‘c’, ‘g’, and T (or ‘u’ in RNA), i.e. to DNA, RNA, or modifications thereof. This polynucleotide molecule can be a naturally occurring polynucleotide molecule or a synthetic polynucleotide molecule or a combination of one or more naturally occurring polynucleotide molecules with one or more synthetic polynucleotide molecules. Also encompassed by this definition are naturally occurring polynucleotide molecules in which one or more nucleotides are changed (e.g. by mutagenesis), deleted, or added. A nucleic acid can either be isolated, or integrated in another nucleic acid, e.g. in an expression cassette, a plasmid, or the chromosome of a host cell. A nucleic acid is characterized by its nucleic acid sequence consisting of individual nucleotides. The term “oligonucleotide” denotes a polymeric molecule consisting of at most 10 individual nucleotides (also called bases) ‘a’, ‘c’, ‘g’, and ‘t’ (or ‘u’ in RNA).

To a person skilled in the art procedures and methods are well known to convert an amino acid sequence, e.g. of a polypeptide, into a corresponding nucleic acid sequence encoding this amino acid sequence. Therefore, a nucleic acid is characterized by its nucleic acid sequence consisting of individual nucleotides and likewise by the amino acid sequence of a polypeptide encoded thereby.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “recombinant polypeptide” and “recombinantly produced polypeptide” denote a polypeptide that is prepared, expressed, or created by recombinant means, such as polypeptides isolated from host cells, such as E. coli, NS0, BHK, or CHO cells.

The term “substituting” denotes the change of one specific nucleotide in a parent nucleic acid to obtain a substituted/changed nucleic acid.

The method as reported herein:

Methods and techniques known to a person skilled in the art, which are useful for carrying out the current invention, are described e.g. in Ausubel, F. M., et al. (eds.), Current Protocols in Molecular Biology, Volumes I to III, John Wiley and Sons, Inc., New York (1997); Sambrook, J., et al., Molecular Cloning: A Laboratory Manual, Second Edition, Cold Spring Harbor Laboratory Press, Cold Spring Harbor, N.Y. (1989), Morrison, S. L., et al., Proc. Natl. Acad. Sci. USA 81 (1984) 6851-6855; U.S. Pat. No. 5,202,238 and U.S. Pat. No. 5,204,244.

For each organism a characteristic (individual) usage of codons for encoding defined amino acids can be given. For example the amino acid glutamine (Q in one letter code) can be encoded by two different codons (due to the degeneracy of the genetic code), i.e. cag and caa. In humans the two glutamine codons have a usage frequency of 74% and 26%, respectively. In E. coli the usage frequency is comparable, i.e. 82% and 18%, respectively. The amino acid lysine (K) can also be encoded by two different codon, i.e. aag and aaa. In humans the two different lysine encoding codons have a usage frequency of 59% and 41%, respectively, whereas in E. coli the two different lysine encoding codons have a non-even usage frequency of 20% and 80%, respectively. It has been found that the oligonucleotide that encodes the tripeptide QKK which is comprised in a nucleic acid encoding a polypeptide that comprises the tripeptide QKK can be the point of a 1->3 frameshift (mutation) during the transcription or translation process of the nucleic acid that encodes the polypeptide which comprises the tripeptide QKK. Due to the occurrence of the frameshift a polypeptide with a not-encoded amino acid sequence, most probably a nonsense or shortened amino acid sequence, is produced.

In more detail, it has been found that depending on the oligonucleotide, which encodes the tripeptide QKK and which is comprised in a larger, i.e. an at least 50 amino acid residue, polypeptide encoding nucleic acid, a 1->3 frameshift during the transcription or translation process of the oligonucleotide occurs. The frequency of the frameshift is depending on the combination of individual codons (see the following Table).

TABLE

| QKK tripeptide encoding | 1→3 frameshift |
|---|---|
| oligonucleotide | occurrence |
| caa aaa aag | 10% |
| (SEQ ID NO: 01) | |
| caa aag aag | 30% |
| (SEQ ID NO: 02) | |
| cag aag aag | below detection limit |
| (SEQ ID NO: 03) | |
| caa aag aaa | below detection limit |
| (SEQ ID NO: 04) | |
| cag aaa aaa | below detection limit |
| (SEQ ID NO: 05) | |

It can be seen that in E. coli a 1->3 frameshift occurs if the tripeptide QKK is encoded by the nucleic acids caa aaa aag and caa aag aag. It has now surprisingly been found that this frameshift can be prevented by using the nucleic acid sequences cag aag aag (SEQ ID NO: 03), or caa aag aaa (SEQ ID NO: 04), or cag aaa aaa (SEQ ID NO: 05). Thus, the expression yield of full length polypeptide can be improved (likewise the formation of non-full length polypeptide by-products can be reduced) by using a nucleic acid of SEQ ID NO: 03, or SEQ ID NO: 04, or SEQ ID NO: 05 for encoding the tripeptide QKK in the polypeptide.

Thus, one aspect as reported herein is a method for the recombinant production of a (full length) polypeptide in E. coli, which comprises the tripeptide QKK (SEQ ID NO: 06), characterized in that the method comprises the following step:
recovering the polypeptide from the cells or the cultivation medium of a cultivation of a cell comprising a nucleic acid encoding the polypeptide and thereby producing the polypeptide,
   whereby the tripeptide QKK comprised in the polypeptide is encoded by the oligonucleotide cag aag aag (SEQ ID NO: 03), or the oligonucleotide caa aag aaa (SEQ ID NO: 04), or the oligonucleotide cag aaa aaa (SEQ ID NO: 05).

Thus, one aspect as reported herein is a method for the recombinant production of a (full length) polypeptide in E. coli, which comprises the tripeptide QKK (SEQ ID NO: 06), characterized in that the method comprises the following step:
recovering the polypeptide from the cells or the cultivation medium of a cultivation of a cell comprising a nucleic acid encoding the polypeptide and thereby producing the polypeptide,
   whereby the tripeptide QKK comprised in the polypeptide is encoded by the oligonucleotide the oligonucleotide caa aag aaa (SEQ ID NO: 04), or the oligonucleotide cag aaa aaa (SEQ ID NO: 05).

In one embodiment the method comprises the following steps:
providing a cell comprising a nucleic acid encoding the polypeptide,
cultivating the cell (under conditions which are suitable for the expression of the polypeptide),
recovering the polypeptide from the cell or the cultivation medium.
optionally purifying the produced polypeptide with one or more chromatography steps.

In one embodiment the polypeptide encoding nucleic acid comprising the tripeptide QKK encoding oligonucleotide cag aag aag (SEQ ID NO: 03), or the oligonucleotide caa aag aaa (SEQ ID NO: 04), or the oligonucleotide cag aaa aaa (SEQ ID NO: 05) is obtained by substituting one to three nucleotides in the tripeptide QKK encoding oligonucleotide caa aaa aag (SEQ ID NO. 01), or the oligonucleotide caa aag aag (SEQ ID NO: 02) to obtain the oligonucleotide cag aag aag (SEQ ID NO: 03), or the oligonucleotide caa aag aaa (SEQ ID NO: 04), or the oligonucleotide cag aaa aaa (SEQ ID NO: 05).

In one embodiment the produced polypeptide is purified with one to five chromatography steps.

In one embodiment the produced polypeptide is purified with two to four chromatography steps.

In one embodiment the produced polypeptide is purified with three chromatography steps.

General chromatographic methods and their use are known to a person skilled in the art. See for example, Heftmann, E. (ed.), Chromatography, 5^{th} edition, Part A: Fundamentals and Techniques, Elsevier Science Publishing Company, New York (1992); Deyl, Z. (ed.), Advanced Chromatographic and Electromigration Methods in Biosciences, Elsevier Science BV, Amsterdam, The Netherlands (1998); Poole, C. F., and Poole, S. K., Chromatography Today, Elsevier Science Publishing Company, New York (1991); Scopes, R. K., Protein Purification: Principles and Practice (1982); Sambrook, J., et al. (ed.), Molecular Cloning: A Laboratory Manual, Second Edition, Cold Spring Harbor Laboratory Press, Cold Spring Harbor, N.Y. (1989); or Ausubel, F. M., et al. (eds.), Current Protocols in Molecular Biology, Volumes I to III, John Wiley & Sons, Inc., New York (1997).

One aspect as reported herein is a nucleic acid encoding a polypeptide that comprises the tripeptide QKK in its amino acid sequence, whereby the tripeptide QKK is encoded by the oligonucleotide caa aag aaa (SEQ ID NO: 04), or the oligonucleotide cag aaa aaa (SEQ ID NO: 05).

One aspect as reported herein is a cell comprising a nucleic acid as reported herein.

One aspect as reported herein is the use of the oligonucleotide caa aag aaa (SEQ ID NO: 04), or the oligonucleotide cag aaa aaa (SEQ ID NO: 05) for encoding the tripeptide QKK comprised in a polypeptide.

One aspect as reported herein is a method for reducing the by-product formation during the recombinant production of a (full length) polypeptide in E. coli, which comprises the tripeptide QKK, comprising the step of:
substituting in the polypeptide encoding nucleic acid one to three nucleotides in the tripeptide QKK encoding oligonucleotide caa aaa aag (SEQ ID NO. 01), or the oligonucleotide caa aag aag (SEQ ID NO: 02) to obtain the oligonucleotide cag aag aag (SEQ ID NO: 03), or the oligonucleotide caa aag aaa (SEQ ID NO: 04), or the oligonucleotide cag aaa aaa (SEQ ID NO: 05), thereby producing a substituted polypeptide encoding nucleic acid, and
recovering the polypeptide from the cells or the cultivation medium of a cultivation of a cell comprising the substituted nucleic acid encoding the polypeptide and thereby reducing the by-product formation during the recombinant production of a polypeptide, which comprises the tripeptide QKK.

One aspect as reported herein is a method for reducing the by-product formation during the recombinant production of a (full length) polypeptide in E. coli, which comprises the tripeptide QKK, comprising the step of:
substituting in the polypeptide encoding nucleic acid one to three nucleotides in the tripeptide QKK encoding oligonucleotide caa aaa aag (SEQ ID NO. 01), or the oligonucleotide caa aag aag (SEQ ID NO: 02) to obtain the oligonucleotide caa aag aaa (SEQ ID NO: 04) or the oligonucleotide cag aaa aaa (SEQ ID NO: 05), thereby producing a substituted polypeptide encoding nucleic acid, and
recovering the polypeptide from the cells or the cultivation medium of a cultivation of a cell comprising the substituted nucleic acid encoding the polypeptide and thereby reducing the by-product formation during the recombinant production of a polypeptide, which comprises the tripeptide QKK.

One aspect as reported herein is a method for increasing the expression of a recombinantly produced (full length) polypeptide in E. coli, which comprises the tripeptide QKK, comprising the step of:
substituting in the polypeptide encoding nucleic acid one to three nucleotides in the tripeptide QKK encoding oligonucleotide caa aaa aag (SEQ ID NO. 01), or the oligonucleotide caa aag aag (SEQ ID NO: 02) to obtain the oligonucleotide cag aag aag (SEQ ID NO: 03), or the oligonucleotide caa aag aaa (SEQ ID NO: 04), or the oligonucleotide cag aaa aaa (SEQ ID NO: 05), thereby producing a substituted polypeptide encoding nucleic acid, and
recovering the polypeptide from the cells or the cultivation medium of a cultivation of a cell comprising the substituted nucleic acid encoding the polypeptide and thereby increasing the expression of the polypeptide.

One aspect as reported herein is a method for increasing the expression of a recombinantly produced (full length) polypeptide in E. coli, which comprises the tripeptide QKK, comprising the step of:
substituting in the polypeptide encoding nucleic acid one to three nucleotides in the tripeptide QKK encoding oligonucleotide caa aaa aag (SEQ ID NO. 01), or the oligonucleotide caa aag aag (SEQ ID NO: 02), or the oligonucleotide cag aag aag (SEQ ID NO: 03) to obtain the oligonucleotide caa aag aaa (SEQ ID NO: 04), or the oligonucleotide cag aaa aaa (SEQ ID NO: 05), thereby producing a substituted polypeptide encoding nucleic acid, and
recovering the polypeptide from the cells or the cultivation medium of a cultivation of a cell comprising the substituted nucleic acid encoding the polypeptide and thereby increasing the expression of the polypeptide.

In one embodiment of each of the individual previous aspects the method comprises one or more of the following further steps:
providing the amino acid sequence or the encoding nucleic acid of a polypeptide comprising the tripeptide QKK, and/or
transfecting a cell with the substituted nucleic acid encoding the polypeptide, and/or
cultivating the cell transfected with the substituted nucleic acid (under conditions which are suitable for the expression of the polypeptide), and/or
recovering the polypeptide from the cell or the cultivation medium, and/or
optionally purifying the produced polypeptide with one or more chromatography steps.

In one embodiment the produced polypeptide is purified with one to five chromatography steps.

In one embodiment the produced polypeptide is purified with two to four chromatography steps.

In one embodiment the produced polypeptide is purified with three chromatography steps.

The method as reported herein is exemplified in the following with a recombinant polypeptide produced in a prokaryotic cell, i.e. a tetranectin-apolipoprotein A-I fusion polypeptide produced in E. coli.

The tetranectin-apolipoprotein A-I fusion polypeptide comprises (in N- to C-terminal direction) the human tetranectin trimerising structural element and wild-type human apolipoprotein A-I. The amino acid sequence of the human tetranectin trimerising structural element can be shortened by the first 9 amino acids, thus, starting with the isoleucine residue of position 10, a naturally occurring truncation site. As a consequence of this truncation the O-glycosylation site at threonine residue of position 4 has been deleted. Between the tetranectin trimerising structural element and the human apolipoprotein A-I the five amino acid residues SLKGS (SEQ ID NO: 08) were removed.

For improved expression and purification a construct can be generated comprising an N-terminal purification tag, e.g. a hexahistidine-tag, and a protease cleavage site for removal of the purification tag. In one embodiment the protease is IgA protease and the protease cleavage site is an IgA protease cleavage site. As a result of the specific cleavage of the protease some amino acid residues of the protease cleavage site are retained at the N-terminus of the polypeptide, i.e. in case of an IgA protease cleavage site two amino acid residues—as first alanine or glycine or serine or threonine and as second proline—are maintained at the N-terminus of the polypeptide, e.g. the tetranectin-apolipoprotein A-I fusion polypeptide.

The tetranectin trimerising structural element provides for a domain that allows for the formation of a tetranectin-apolipoprotein A-I homo-trimer that is constituted by non-covalent interactions between each of the individual tetranectin-apolipoprotein A-I monomers.

In one embodiment the apolipoprotein A-I fusion polypeptide is a variant comprising conservative amino acid substitutions.

In one embodiment the tetranectin-apolipoprotein A-I fusion polypeptide comprises an expression and purification tag and has the amino acid sequence of

(SEQ ID NO: 09)
CDLPQTHSLGSHHHHHHGSVVAPPAPIVNAKKDVVNTKMFEELKSR
LDTLAQEVALLKEQQALQTVDEPPQSPWDRVKDLATVYVDVLKDSG
RDYVSQFEGSALGKQLNLKLLDNWDSVTSTFSKLREQLGPVTQEFW
DNLEKETEGLRQEMSKDLEEVKAKVQPYLDDFQKKWQEEMELYRQK
VEPLRAELQEGARQKLHELQEKLSPLGEEMRDRARAHVDALRTHLA
PYSDELRQRLAARLEALKENGGARLAEYHAKATEHLSTLSEKAKPA
LEDLRQGLLPVLESFKVSFLSALEEYTKKLNTQ.

In one embodiment the tetranectin-apolipoprotein A-I fusion polypeptide (IVN) has the amino acid sequence of

(SEQ ID NO: 10)
IVNAKKDVVNTKMFEELKSRLDTLAQEVALLKEQQALQTVDEPPQS
PWDRVKDLATVYVDVLKDSGRDYVSQFEGSALGKQLNLKLLDNWDS
VTSTFSKLREQLGPVTQEFWDNLEKETEGLRQEMSKDLEEVKAKVQ
PYLDDFQKKWQEEMELYRQKVEPLRAELQEGARQKLHELQEKLSPL
GEEMRDRARAHVDALRTHLAPYSDELRQRLAARLEALKENGGARLA
EYHAKATEHLSTLSEKAKPALEDLRQGLLPVLESFKVSFLSALEEY
TKKLNTQ.

Thus, in one preferred embodiment the tetranectin-apolipoprotein A-I fusion polypeptide (PIVN) has the amino acid sequence of

(SEQ ID NO: 11)
PIVNAKKDVVNTKMFEELKSRLDTLAQEVALLKEQQALQTVDEPPQ
SPWDRVKDLATVYVDVLKDSGRDYVSQFEGSALGKQLNLKLLDNWD
SVTSTFSKLREQLGPVTQEFWDNLEKETEGLRQEMSKDLEEVKAKV
QPYLDDFQKKWQEEMELYRQKVEPLRAELQEGARQKLHELQEKLSP
LGEEMRDRARAHVDALRTHLAPYSDELRQRLAARLEALKENGGARL
AEYHAKATEHLSTLSEKAKPALEDLRQGLLPVLESFKVSFLSALEE
YTKKLNTQ.

In one embodiment the tetranectin-apolipoprotein A-I fusion polypeptide (XPIVN) has the amino acid sequence of

(SEQ ID NO: 12)
(G,S,T)PIVNAKKDVVNTKMFEELKSRLDTLAQEVALLKEQQALQ
TVDEPPQSPWDRVKDLATVYVDVLKDSGRDYVSQFEGSALGKQLNL
KLLDNWDSVTSTFSKLREQLGPVTQEFWDNLEKETEGLRQEMSKDL
EEVKAKVQPYLDDFQKKWQEEMELYRQKVEPLRAELQEGARQKLHE
LQEKLSPLGEEMRDRARAHVDALRTHLAPYSDELRQRLAARLEALK
ENGGARLAEYHAKATEHLSTLSEKAKPALEDLRQGLLPVLESFKVS
FLSALEEYTKKLNTQ.

Thus, in one embodiment the tetranectin-apolipoprotein A-I fusion polypeptide (APIVN) has the amino acid sequence of

(SEQ ID NO: 13)
APIVNAKKDVVNTKMFEELKSRLDTLAQEVALLKEQQALQTVDEPP
QSPWDRVKDLATVYVDVLKDSGRDYVSQFEGSALGKQLNLKLLDNW
DSVTSTFSKLREQLGPVTQEFWDNLEKETEGLRQEMSKDLEEVKAK
VQPYLDDFQKKWQEEMELYRQKVEPLRAELQEGARQKLHELQEKLS
PLGEEMRDRARAHVDALRTHLAPYSDELRQRLAARLEALKENGGAR
LAEYHAKATEHLSTLSEKAKPALEDLRQGLLPVLESFKVSFLSALE
EYTKKLNTQ.

In one embodiment the tetranectin-apolipoprotein A-I fusion polypeptide (XIVN) comprising a hexa-histidine-tag has the amino acid sequence of

(SEQ ID NO: 14)
HHHHHHXIVNAKKDVVNTKMFEELKSRLDTLAQEVALLKEQQALQT
VDEPPQSPWDRVKDLATVYVDVLKDSGRDYVSQFEGSALGKQLNLK
LLDNWDSVTSTFSKLREQLGPVTQEFWDNLEKETEGLRQEMSKDLE
EVKAKVQPYLDDFQKKWQEEMELYRQKVEPLRAELQEGARQKLHEL
QEKLSPLGEEMRDRARAHVDALRTHLAPYSDELRQRLAARLEALKE
NGGARLAEYHAKATEHLSTLSEKAKPALEDLRQGLLPVLESFKVSF
LSALEEYTKKLNTQ,

wherein X can be any of the following amino acid sequences A, G, S, P, AP, GP, SP, PP, GSAP (SEQ ID NO: 15), GSGP (SEQ ID NO: 16), GSSP (SEQ ID NO: 17), GSPP (SEQ ID NO: 18), GGGS (SEQ ID NO: 19), GGGGS (SEQ ID NO: 20), GGGSGGGS (SEQ ID NO: 21), GGGGSGGGGS (SEQ ID NO: 22), GGGSGGGSGGGS (SEQ ID NO: 23), GGGGSGGGGSGGGGS (SEQ ID NO: 24), GGGSAP (SEQ ID NO: 25), GGGSGP (SEQ ID NO: 26), GGGSSP (SEQ ID NO: 27), GGGSPP (SEQ ID NO: 28), GGGGSAP (SEQ ID NO: 29), GGGGSGP (SEQ ID NO: 30), GGGGSSP (SEQ ID NO: 31), GGGGSPP (SEQ ID NO: 32), GGGSGGGSAP (SEQ ID NO: 33), GGGSGGGSGP (SEQ ID NO: 34), GGGSGGGSSP (SEQ ID NO: 35), GGGSGGGSPP (SEQ ID NO: 36), GGGSGGGSGGGSAP (SEQ ID NO: 37), GGGSGGGSGGGSGP (SEQ ID NO: 38), GGGSGGGSGGGSSP (SEQ ID NO: 39), GGGSGGGSGGGSPP (SEQ ID NO: 40), GGGGSAP (SEQ ID NO: 41), GGGGSGP (SEQ ID NO: 42), GGGGSSP (SEQ ID NO: 43), GGGGSPP (SEQ ID NO: 44), GGGGSGGGGSAP (SEQ ID NO: 45), GGGGSGGGGSGP (SEQ ID NO: 46), GGGGSGGGGSSP (SEQ ID NO: 47), GGGGSGGGGSPP (SEQ ID NO: 48), GGGGSGGGGSGGGGSAP (SEQ ID NO: 49), GGGGSGGGGSGGGGSGP (SEQ ID NO: 50), GGGGSGGGGSGGGGSSP (SEQ ID NO: 51), and GGGGSGGGGSGGGGSPP (SEQ ID NO: 52).

It has to be noted that if a polypeptide is recombinantly produced in E. coli strains the N-terminal methionine residue is usually not efficiently cleaved off by E. coli proteases. Thus, the N-terminal methionine residue is partially present in the produced polypeptide.

A tetranectin-apolipoprotein A-I fusion polypeptide of SEQ ID NO: 09 was recombinantly produced in E. coli. A main by-product (about 10% of total protein) could be detected.

Via Lys-C peptide mapping (LC-ESI-MS/MS) and top-down MS it was confirmed that the N-terminal amino acid sequence of amino acid residues 1 to 148 (lysine) was correct (as given in SEQ ID NO: 13). The C-terminal amino acid sequence of the shortened by-product was VARRNGTVQTES (SEQ ID NO: 53). The deviation from the sequence of the target tetranectin-apolipoprotein A-I fusion polypeptide started at the tripeptide QKK. The change of the C-terminal amino acid sequence was due to a 1->3 frameshift of the reading frame during the translation or transcription process (see FIG. 1).

Different variants of the oligonucleotide encoding the tripeptide QKK were tested. It has been found that the oligonucleotide caa aag aag (SEQ ID NO: 02) even further increased the amount of the shortened by-product to 30%. In contrast thereto by using the oligonucleotides cag aag aag (SEQ ID NO: 03), caa aag aaa (SEQ ID NO: 04) and cag aaa aaa (SEQ ID NO: 04) the formation of the shortened by-product could be reduced below the detection limit of the employed LC-MS method (see FIG. 2).

The following examples, sequence listing and figures are provided to aid the understanding of the present invention, the true scope of which is set forth in the appended claims. It is understood that modifications can be made in the procedures set forth without departing from the spirit of the invention.

Description of the Sequence Listing

| | |
|---|---|
| SEQ ID | Oligonucleotide caa aaa aag. |
| NO: 01 | |
| SEQ ID | Oligonucleotide caa aag aag. |
| NO: 02 | |
| SEQ ID | Oligonucleotide cag aag aag. |
| NO: 03 | |
| SEQ ID | Oligonucleotide caa aag aaa. |
| NO: 04 | |
| SEQ ID | Oligonucleotide cag aaa aaa. |
| NO: 05 | |
| SEQ ID | Tripeptide QKK. |
| NO: 06 | |
| SEQ ID | Human apolipoprotein A-I. |
| NO: 07 | |
| SEQ ID | Removed SLKGS polypeptide. |
| NO: 08 | |
| SEQ ID | Tetranectin-apolipoprotein A-I fusion |
| NO: 09 | polypeptide comprising expres- |
| | sion and purification tags. |
| SEQ ID | Tetranectin-apolipoprotein A-I fusion |
| NO: 10 | polypeptide (IVN). |
| SEQ ID | Tetranectin-apolipoprotein A-I fusion |
| NO: 11 | polypeptide (PIVN). |
| SEQ ID | Tetranectin-apolipoprotein A-I fusion |
| NO: 12 | polypeptide (XPIVN). |
| SEQ ID | Tetranectin-apolipoprotein A-I fusion |
| NO: 13 | polypeptide (APIVN). |
| SEQ ID | Tetranectin-apolipoprotein A-I fusion |
| NO: 14 | polypeptide (XIVN)comprising |
| | hexa-histidine-tag. |
| SEQ ID | Linker polypeptides. |
| NO: 15 | |
| to 52 | |
| SEQ ID | C-terminal amino acid sequence |
| NO: 53 | of main by-product. |
| SEQ ID | Interferon fragment. |
| NO: 54 | |
| SEQ ID | Hexa-histidine tag. |
| NO: 55 | |
| SEQ ID | IgA protease cleavage site. |
| NO: 56 | |

### DESCRIPTION OF THE FIGURES

FIG. 1 Different reading frames result in different amino acid sequences, whereby a 1->3 frameshift results in a shortened product (ΔMW=−14369 Da) with the determined C-terminal amino acid sequence.

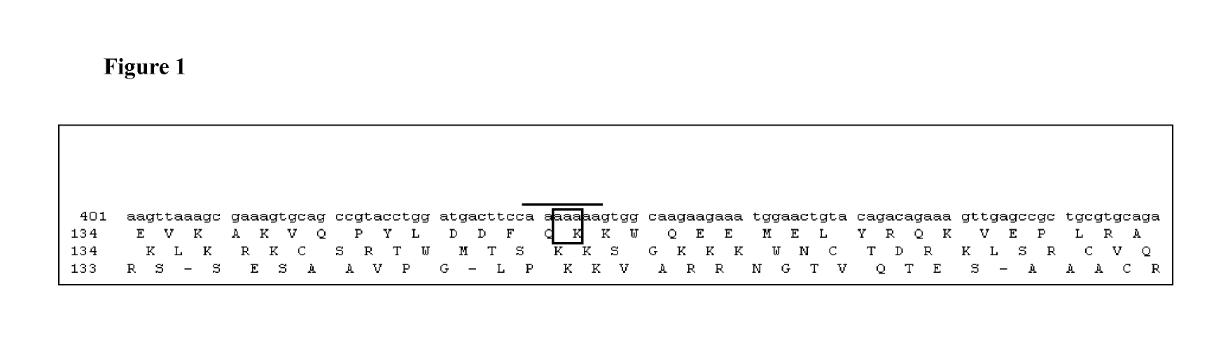

FIG. 2 LC-MS analysis of constructs comprising different oligonucleotides encoding the tripeptide QKK with respect to formation of 1->3 frameshift by-product.

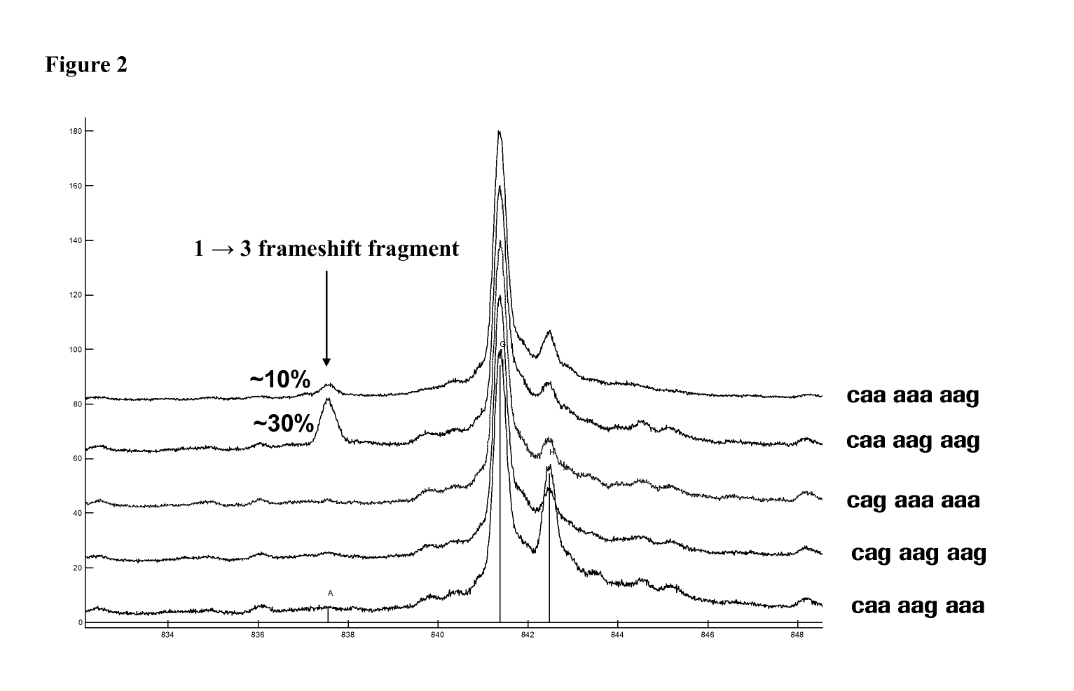

### MATERIALS AND METHODS

### Protein Determination:

The protein concentration was determined by determining the optical density (OD) at 280 nm, using the molar extinction coefficient calculated on the basis of the amino acid sequence.

### Recombinant DNA Technique:

Standard methods were used to manipulate DNA as described in Sambrook, J., et al., Molecular Cloning: A Laboratory Manual, Second Edition, Cold Spring Harbor Laboratory Press, Cold Spring Harbor, N.Y. (1989). The molecular biological reagents were used according to the manufacturer's instructions.

### Example 1

### Making and Description of the E. coli Expression Plasmids

The tetranectin-apolipoprotein A-I fusion polypeptide was prepared by recombinant means. The amino acid sequence of the expressed fusion polypeptide in N- to C-terminal direction is as follows:
the amino acid methionine (M),
a fragment of an interferon sequence that has the amino acid sequence of CDLPQTHSL (SEQ ID NO: 54),
a GS linker,
a hexa-histidine tag that has the amino acid sequence of HHHHHH (SEQ ID NO: 55),
a GS linker,
an IgA protease cleavage site that has the amino acid sequence of VVAPPAP (SEQ ID NO: 56), and
a tetranectin-apolipoprotein A-I that has the amino acid sequence of SEQ ID NO: 10.

The tetranectin-apolipoprotein A-I fusion polypeptide as described above is a precursor polypeptide from which the final tetranectin-apolipoprotein A-I fusion polypeptides was released by enzymatic cleavage in vitro using IgA protease.

The precursor polypeptide encoding fusion gene was assembled with known recombinant methods and techniques by connection of appropriate nucleic acid segments. Nucleic acid sequences made by chemical synthesis were verified by DNA sequencing. The expression plasmid for the production of tetranectin-apolipoprotein A-I fusion polypeptide of SEQ ID NO: 10 encoding a fusion polypeptide of SEQ ID NO: 09 was prepared as follows.

Making of the E. coli Expression Plasmid:

Plasmid 4980 (4980-pBRori-URA3-LACI-SAC) is an expression plasmid for the expression of core-streptavidin in E. coli. It was generated by ligation of the 3142 bp long EcoRI/CelII-vector fragment derived from plasmid 1966 (1966-pBRori-URA3-LACI-T-repeat; reported in EP-B 1 422 237) with a 435 bp long core-streptavidin encoding EcoRI/CelII-fragment.

The core-streptavidin E. coli expression plasmid comprises the following elements:
the origin of replication from the vector pBR322 for replication in E. coli (corresponding to by position 2517-3160 according to Sutcliffe, G., et al., Quant. Biol. 43 (1979) 77-90),
the URA3 gene of Saccharomyces cerevisiae coding for orotidine 5′-phosphate decarboxylase (Rose, M., et al., Gene 29 (1984) 113-124) which allows plasmid selection by complementation of E. coli pyrF mutant strains (uracil auxotrophy),
the core-streptavidin expression cassette comprising
   the T5 hybrid promoter (T5-PN25/03/04 hybrid promoter according to Bujard, H., et al., Methods. Enzymol. 155 (1987) 416-433 and Stueber, D., et al., Immunol. Methods IV (1990) 121-152) including a synthetic ribosomal binding site according to Stueber, D., et al. (see before),
   the core-streptavidin gene,
   two bacteriophage-derived transcription terminators, the λ-T0 terminator (Schwarz, E., et al., Nature 272 (1978) 410-414) and the fd-terminator (Beck, E. and Zink, B., Gene 1-3 (1981) 35-58),
the lacI repressor gene from E. coli (Farabaugh, P. J., Nature 274 (1978) 765-769).

The final expression plasmid for the expression of the tetranectin-apolipoprotein A-I precursor polypeptide was prepared by excising the core-streptavidin structural gene from vector 4980 using the singular flanking EcoRI and CelII restriction endonuclease cleavage site and inserting the EcoRII/CelII restriction site flanked nucleic acid encoding the precursor polypeptide into the 3142 bp long EcoRI/CelII-4980 vector fragment.

### Example 2

### Expression of Tetranectin-Apolipoprotein A-I

For the expression of the fusion protein there was employed an E. coli host/vector system which enables an antibiotic-free plasmid selection by complementation of an E. coli auxotrophy (PyrF) (see EP 0 972 838 and U.S. Pat. No. 6,291,245).

The E. coli K12 strain CSPZ-2 (leuB, proC, trpE, th-1, ΔpyrF) was transformed by electroporation with the expression plasmid p(IFN-His6-IgA-tetranectin-apolipoprotein A-I). The transformed E. coli cells were first grown at 37° C. on agar plates.

### Fermentation Protocol 1:

For pre-fermentation a M9 medium according to Sambrook, J., et al. (Molecular Cloning: A Laboratory Manual, Second Edition, Cold Spring Harbor Laboratory Press, Cold Spring Harbor, N.Y. (1989)) supplemented with about 1 g/l L-leucine, about 1 g/l L-proline and about 1 mg/l thiamine-HCl has been used.

For pre-fermentation 300 ml of M9-medium in a 1000 ml Erlenmeyer-flask with baffles was inoculated with 2 ml out of a primary seed bank ampoule. The cultivation was performed on a rotary shaker for 13 hours at 37° C. until an optical density (578 nm) of 1-3 was obtained.

For fermentation a batch medium according to Riesenberg, et al. was used (Riesenberg, D., et al., J. Biotechnol. 20 (1991) 17-27): 27.6 g/l glucose*H₂O, 13.3 g/l KH₂PO₄, 4.0 g/l (NH₄)₂HPO₄, 1.7 g/l citrate, 1.2 g/l MgSO₄*7 H₂O, 60 mg/l iron(III)citrate, 2.5 mg/l CoCl₂*6 H₂O, 15 mg/l MnCl₂*4 H₂O, 1.5 mg/l CuCl₂*2 H₂O, 3 mg/l H₃BO₃, 2.5 mg/l Na₂MoO₄*2 H₂O, 8 mg/l Zn(CH₃COO)₂*2 H₂O, 8.4 mg/l Titriplex III, 1.3 ml/l Synperonic 10% anti foam agent. The batch medium was supplemented with 5.4 mg/l thiamin-HCl and 1.2 g/l L-leucine and L-proline respectively. The feed 1 solution contained 700 g/l glucose supplemented with 19.7 g/l MgSO₄*7 H₂O. The alkaline solution for pH regulation was an aqueous 12.5% (w/v) NH₃ solution supplemented with 50 g/l L-leucine and 50 g/l L-proline respectively. All components were dissolved in deionized water.

The fermentation was carried out in a 10 l Biostat C DCU3 fermenter (Sartorius, Melsungen, Germany). Starting with 6.4 l sterile fermentation batch medium plus 300 ml inoculum from the pre-fermentation the batch fermentation was performed at 37° C., pH 6.9±0.2, 500 mbar and an aeration rate of 10 l/min. After the initially supplemented glucose was depleted the temperature was shifted to 28° C. and the fermentation entered the fed-batch mode. Here the relative value of dissolved oxygen (pO₂) was kept at 50% (DO-stat, see e.g. Shay, L. K., et al., J. Indus. Microbiol. Biotechnol. 2 (1987) 79-85) by adding feed 1 in combination with constantly increasing stirrer speed (550 rpm to 1000 rpm within 10 hours and from 1000 rpm to 1400 rpm within 16 hours) and aeration rate (from 10 l/min to 16 l/min in 10 hours and from 16 l/min to 20 l/min in 5 hours). The supply with additional amino acids resulted from the addition of the alkaline solution, when the pH reached the lower regulation limit (6.70) after approximately 8 hours of cultivation. The expression of recombinant therapeutic protein was induced by the addition of 1 mM IPTG at an optical density of 70.

At the end of fermentation the cytoplasmatic and soluble expressed tetranectin-apolipoprotein A-I is transferred to insoluble protein aggregates, the so called inclusion bodies, with a heat step where the whole culture broth in the fermenter is heated to 50° C. for 1 or 2 hours before harvest (see e.g. EP-B 1 486 571). Thereafter, the content of the fermenter was centrifuged with a flow-through centrifuge (13,000 rpm, 13 l/h) and the harvested biomass was stored at −20° C. until further processing. The synthesized tetranectin-apolipoprotein A-I precursor proteins were found exclusively in the insoluble cell debris fraction in the form of insoluble protein aggregates, so-called inclusion bodies (IBs).

Samples drawn from the fermenter, one prior to induction and the others at dedicated time points after induction of protein expression are analyzed with SDS-Polyacrylamide gel electrophoresis.

From every sample the same amount of cells (OD_{Target}=5) are resuspended in 5 mL PBS buffer and disrupted via sonication on ice. Then 100 μL of each suspension are centrifuged (15,000 rpm, 5 minutes) and each supernatant is withdrawn and transferred to a separate vial. This is to discriminate between soluble and insoluble expressed target protein. To each supernatant (=soluble) fraction 300 μL and to each pellet (=insoluble) fraction 400 μL of SDS sample buffer (Laemmli, U. K., Nature 227 (1970) 680-685) are added. Samples are heated for 15 minutes at 95° C. under shaking to solubilize and reduce all proteins in the samples. After cooling to room temperature 5 μL of each sample are transferred to a 4-20% TGX Criterion Stain Free polyacrylamide gel (Bio-Rad). Additionally 5 μl molecular weight standard (Precision Plus Protein Standard, Bio-Rad) and 3 amounts (0.3 μl, 0.6 μl and 0.9 μl) quantification standard with known product protein concentration (0.1 μg/μl) are positioned on the gel.

The electrophoresis was run for 60 Minutes at 200 V and thereafter the gel was transferred the GelDOC EZ Imager (Bio-Rad) and processed for 5 minutes with UV radiation. Gel images were analyzed using Image Lab analysis software (Bio-Rad). With the three standards a linear regression curve was calculated with a coefficient of >0.99 and thereof the concentrations of target protein in the original sample was calculated.

### Fermentation Protocol 2:

For pre-fermentation a M9 medium according to Sambrook, J., et al. (Molecular Cloning: A Laboratory Manual, Second Edition, Cold Spring Harbor Laboratory Press, Cold Spring Harbor, N.Y. (1989)) supplemented with about 1 g/l L-leucine, about 1 g/l L-proline and about 1 mg/l thiamine-HCl has been used.

For pre-fermentation 300 ml of modified M9-medium in a 1000 ml Erlenmeyer-flask with baffles was inoculated from agar plate or with 1-2 ml out of a primary seed bank ampoule. The cultivation was performed on a rotary shaker for 13 hours at 37° C. until an optical density (578 nm) of 1-3 was obtained.

For fermentation and high yield expression of tetranectin-apolipoprotein A-I the following batch medium and feeds were used:

8.85 g/l glucose, 63.5 g/l yeast extract, 2.2 g/l NH₄C1, 1.94 g/l L-leucine, 2.91 g/l L-proline, 0.74 g/l L-methionine, 17.3 g/l KH₂PO₄*H2_{O}, 2.02 g/l MgSO₄*7 H₂O, 25.8 mg/l thiamin-HCl, 1.0 ml/l Synperonic 10% anti foam agent. The feed 1 solution contained 333 g/l yeast extract and 333 g/l 85%-glycerol supplemented with 1.67 g/l L-methionine and 5 g/l L-leucine and L-proline each. The feed 2 was a solution of 600 g/l L-Proline. The alkaline solution for pH regulation was a 10% (w/v) KOH solution and as acid a 75% glucose solution was used. All components were dissolved in deionized water.

The fermentation was carried out in a 10 l Biostat C DCU3 fermenter (Sartorius, Melsungen, Germany). Starting with 5.15 l sterile fermentation batch medium plus 300 ml inoculum from the pre-fermentation the fed-batch fermentation was performed at 25° C., pH 6.7±0.2, 300 mbar and an aeration rate of 10 l/min. Before the initially supplemented glucose was depleted the culture reached an optical density of 15 (578 nm) and the fermentation entered the fed-batch mode when feed 1 was started with 70 g/h. Monitoring the glucose concentration in the culture the feed 1 was increased to a maximum of 150 g/h while avoiding glucose accumulation and keeping the pH near the upper regulation limit of 6.9. At an optical density of 50 (578 nm) feed 2 was started with a constant feed rate of 10 ml/h. The relative value of dissolved oxygen (pO₂) was kept above 50% by increasing stirrer speed (500 rpm to 1500 rpm), aeration rate (from 10 l/min to 20 l/min) and pressure (from 300 mbar to 500 mbar) in parallel. The expression of recombinant therapeutic protein was induced by the addition of 1 mM IPTG at an optical density of 90.

Seven samples drawn from the fermenter, one prior to induction and the others at dedicated time points after induction of protein expression are analyzed with SDS-Polyacrylamide gel electrophoresis. From every sample the same amount of cells (OD_{Target}=5) are resuspended in 5 mL PBS buffer and disrupted via sonication on ice. Then 100 μL of each suspension are centrifuged (15,000 rpm, 5 minutes) and each supernatant is withdrawn and transferred to a separate vial. This is to discriminate between soluble and insoluble expressed target protein. To each supernatant (=soluble) fraction 300 μL and to each pellet (=insoluble) fraction 200 μL of SDS sample buffer (Laemmli, U. K., Nature 227 (1970) 680-685) are added. Samples are heated for 15 minutes at 95° C. under shaking to solubilize and reduce all proteins in the samples. After cooling to room temperature 5 μL of each sample are transferred to a 10% Bis-Tris polyacrylamide gel (Novagen). Additionally 5 μL molecular weight standard (Precision Plus Protein Standard, Bio-Rad) and 3 amounts (0.3 μl, 0.6 μl and 0.9 μl) quantification standard with known product protein concentration (0.1 μg/μl) are positioned on the gel.

The electrophoresis was run for 35 minutes at 200 V and then the gel was stained with Coomassie Brilliant Blue R dye, destained with heated water and transferred to an optical densitometer for digitalization (GS710, Bio-Rad). Gel images were analyzed using Quantity One 1-D analysis software (Bio-Rad). With the three standards a linear regression curve is calculated with a coefficient of >0.98 and thereof the concentrations of target protein in the original sample was calculated.

At the end of fermentation the cytoplasmatic and soluble expressed tetranectin-apolipoprotein A-I is transferred to insoluble protein aggregates, the so called inclusion bodies (IBs), with a heat step where the whole culture broth in the fermenter is heated to 50° C. for 1 or 2 hours before harvest (see e.g. EP-B 1 486 571). After the heat step the synthesized tetranectin-apolipoprotein A-I precursor proteins were found exclusively in the insoluble cell debris fraction in the form of IBs.

The contents of the fermenter are cooled to 4-8° C., centrifuged with a flow-through centrifuge (13,000 rpm, 13 l/h) and the harvested biomass is stored at −20° C. until further processing. The total harvested biomass yield ranged between 39 g/l and 90 g/l dry matter depending on the expressed construct.

### Example 3

### Preparation of Tetranectin-Apolipoprotein A-I

Inclusion body preparation was carried out by resuspension of harvested bacteria cells in a potassium phosphate buffer solution (0.1 M, supplemented with 1 mM MgSO₄, pH 6.5). After the addition of DNAse the cell were disrupted by homogenization at a pressure of 900 bar. A buffer solution comprising 1.5 M NaCl was added to the homogenized cell suspension. After the adjustment of the pH value to 5.0 with 25% (w/v) HCl the final inclusion body slurry was obtained after a further centrifugation step. The slurry was stored at −20° C. in single use, sterile plastic bags until further processing.

7 g inclusion bodies were solubilized overnight in 140 ml solubilization buffer (8 M guanidinium chloride, 50 mM Tris, 10 mM methionine, pH 8). After centrifugation to remove insoluble material the buffer was changed by diafiltration to 7.2 M guanidinium chloride, 50 mM Tris, 10 mM methionine, pH 8.0 using a SG Hydrosart 10 kDa membrane (Sartorius Stedim). The solution was diluted to 2 M guanidinium chloride by addition of 50 mM Tris, pH 8.0. After centrifugation the solubilized protein was loaded onto an IMAC (Zn²⁺ loaded Fractogel® EMD Chelat, Merck Chemicals) equilibrated in 2 M guanidinium chloride, 50 mM Tris, 10 mM methionine, pH 8.0. After reaching the baseline the column was washed with 20% ethylene glycol, 50 mM Tris, 10 mM methionine followed by re-equilibration with 1 M Tris, 10 mM methionine, pH 8.0.

On-column IgA protease cleavage was performed overnight with IgA protease in 1 M Tris, pH 8.0 (IgA protease:protein=1:100 w/w). The cleaved tetranectin-apolipoprotein A-I fusion polypeptide was washed out of the column with 1 M Tris, 10 mM methionine, pH 8. Buffer exchange to 7.5 M urea, 20 mM Tris, 10 mM methionine, pH 8.0, was achieved by ultrafiltration. The tetranectin-apolipoprotein A-I fusion polypeptide was loaded onto a Q-Sepharose™ Fast Flow (GE Healthcare) equilibrated in the same buffer. The column was washed with 7.5 M urea, 20 mM Tris, pH 8.0 followed by a salt gradient to 75 mM NaCl in equilibration buffer. As soon as the fusion polypeptide started to elute, the salt concentration was kept constant for 10 column volumes. Afterwards the salt gradient was continued, further elution steps were performed with 250 mM and 500 mM NaCl in the same buffer. Collected fractions were dialyzed against 7.2 M guanidinium chloride, 50 mM Tris, 10 mM methionine, pH 8.0 and kept at 4° C.

### Example 4

### Analytics of Tetranectin-Apolipoprotein A-I Fusion Polypeptides

Pools or fractions from the IMAC (Zn²⁺ loaded Fractogel® EMD Chelat) and the Q-Sepharose™ purification columns were desalted and analyzed by electrospray ionization mass spectrometry (ESI-MS).

Desalting was performed offline by size exclusion chromatography using a HR5/20 column (0.7×22 cm, Amersham Bioscience) packed in house with Sephadex G25 Superfine material (Amersham Bioscience 17-0851-01) and an isocratic elution with 40% acetonitrile, 2% formic acid with a flow of 1 ml/min. The signal was monitored at 280 nm wavelength and the eluting tetranectin-apolipoprotein fusion polypeptide peak was collected manually.

ESI-MS to monitor the presence of the fragment was performed on a Q-Star Elite QTOF mass spectrometer (Applied Biosystems (ABI), Darmstadt, Germany) equipped with a Triversa NanoMate source system (Advion, Ithaka, USA) using a declustering potential of 50 and a focusing potential of 200. 15 scans per 5 seconds were recorded in the m/z range of 700 to 2000.

ESI-MS data were analyzed using two software packages, Analyst (Applied Biosystems (ABI), Darmstadt, Germany) and MassAnalyzer (in-house developed software platform). Mass spectra were checked manually for the presence of signals bearing the molecular mass of the protein fragment resulting from the frameshift at the respective QKK tripeptide encoding oligonucleotide (delta of −14369 Da compared to the expected molecular mass of the full-length fusion polypeptide).

1. A method for the recombinant production of a (full length) polypeptide in an E. coli cell, which comprises the tripeptide QKK, characterized in that the method comprises the following step:
recovering the polypeptide from the cells or the cultivation medium of a cultivation of an E. coli cell comprising a nucleic acid encoding the polypeptide and thereby producing the polypeptide,
whereby the tripeptide QKK comprised in the polypeptide is encoded by the oligonucleotide cag aaa aaa, or the oligonucleotide caa aag aaa.

2. A method for reducing the by-product formation during the recombinant production of a full length polypeptide in E. coli, which comprises the tripeptide QKK, comprising the step of:
substituting in the polypeptide encoding nucleic acid one to three nucleotides in the tripeptide QKK encoding oligonucleotide caa aaa aag (SEQ ID NO. 01), or the oligonucleotide caa aag aag (SEQ ID NO: 02), or the oligonucleotide cag aag aag (SEQ ID NO: 03) to obtain the oligonucleotide caa aag aaa (SEQ ID NO: 04), or the oligonucleotide cag aaa aaa (SEQ ID NO: 05), thereby producing a substituted polypeptide encoding nucleic acid, and
recovering the polypeptide from the cells or the cultivation medium of a cultivation of a cell comprising the substituted nucleic acid encoding the polypeptide and thereby reducing the by-product formation during the recombinant production of a polypeptide, which comprises the tripeptide QKK.

3. The method according to any one of claim 1 or 2, characterized in that the method comprises one or more of the following further steps:
providing the amino acid sequence or the encoding nucleic acid of a polypeptide comprising the tripeptide QKK, and/or
transfecting a cell with the substituted nucleic acid encoding the polypeptide, and/or
cultivating the cell transfected with the substituted nucleic acid (under conditions which are suitable for the expression of the polypeptide), and/or
recovering the polypeptide from the cell or the cultivation medium, and/or
optionally purifying the produced polypeptide with one or more chromatography steps.

4. The method according to any one of claims 2 to 3, characterized in that the produced polypeptide is purified with one to five chromatography steps.

5. The method according to any one of the preceding claims, characterized in that the polypeptide is an apolipoprotein A-I, or a variant thereof, or a fusion polypeptide thereof that has the function of apolipoprotein A-I.

6. The method according to claim 5, characterized in that the polypeptide has an amino acid sequence selected from the group comprising SEQ ID NO: 09 to SEQ ID NO: 14.

7. The method according to any one of claims 5 to 6, characterized in that the polypeptide has the amino acid sequence of SEQ ID NO: 11.
